(19) United States
(12) Patent Application Publication
(10) Pub. No.: US 2025/0186316 A1
(43) Pub. Date: Jun. 12, 2025

(54) Coated Glass Particulate-UV Gel Composition and Method for Use

(71) Applicant: Wella Operations US LLC, Calabasas, CA (US)

(72) Inventors: Chaitrali Gothe, Fremont, CA (US); Xiaoyan Tu, Calabasas, CA (US); Sunan Yuvavanich, Thousand Oaks, CA (US)

(21) Appl. No.: 18/976,926

(22) Filed: Dec. 11, 2024

Related U.S. Application Data
(60) Provisional application No. 63/609,261, filed on Dec. 12, 2023.

(51) Int. Cl. A61K 8/25; A61K 8/04; A61K 8/19; A61Q 3/02

(52) U.S. Cl. CPC A61K 8/25; A61K 8/042; A61K 8/19; A61Q 3/02; A61K 2800/412; A61K 2800/623; A61K 2800/651; A61K 2800/81

(57) ABSTRACT

A resilient, abrasion resistant nail covering is described. The nail covering includes a transformed, solidified UV gel with coated glass microparticles.

### CLAIM OF PRIORITY

This patent application claims the benefit of priority to U.S. Provisional Application Ser. No. 63/609,261, filed Dec. 12, 2023, which is incorporated by reference herein in its entirety.

### FIELD OF INVENTION

The present invention is directed to a composition and corresponding method for developing abrasion resistant nail coating compositions. The composition is directed to a UV curable gel incorporating coated glass microparticles for application as a gel to a nail plate.

### BACKGROUND

Nail coating compositions have engendered an ageless reputation of beauty and elegance. Ancient Chinese dynasties demonstrated beauty through intricate dance with complex hand movements accentuated by nail coatings and extensions. With the advent of the late nineteenth and early twentieth centuries, nail coatings developed a cashet for enhanced beauty and glamor through their riveting brilliance and intricate design. Although some social circles of the time took a de-minimus view of artistic design for nail coatings, they nevertheless continued to care for nails and associated tissues by promoting health benefits. Today's societies embrace not only dazzling colors for lacquer coatings but also champion designs involving multi-colored intricate arrangements and nail extensions such as Ombre, Floral and Dot nails as well as French tips.

This said, today's societies also take a practical view toward the cosmetic and chemical underpinnings of nail coatings. Not only should such beauty and glamor aspects be appropriate and impressive for the particular societal circumstance of the day but they should also be environmentally and medically safe, enable reasonable wear and care properties and involve personal choice.

For nail coatings, wear and care properties have traditionally been enhanced by incorporation of solid polymer particles. The polymer particles such as poly(ethyl methacrylate) (PEMA) or poly(methyl methacrylate) (PMMA) deliver to the nail coatings additional abrasion resistance due to their particulate hardness and extremely dense cross-linked polymer network. Incorporation of polymer particles into a lacquer coating or a curable gel coating also involves at least partial softening and/or dissolution of the polymer network at the surfaces of the polymer particles. This phenomenon enables production of a smooth coating surface that is free of surface undulations, irregularities and bumps that would otherwise result from the presence of hard particles on and in the surface of the coating. This phenomenon also produces polymer portions extending from the particles themselves. These portions entwine with the lacquer and/or curable gel polymers which have been described as contributing to the properties of the nail coatings.

Nevertheless, the nail coatings with incorporated polymer particles do not exhibit the abrasion resistance expected based upon the hardness of the particles themselves. While this lower abrasion resistance may be a result of the particle softening, the overall resistance of the coating ought to be enhanced by the incorporation of the polymer portions throughout the coating. Attempts have been made to alter the abrasion resistance of such coatings by changing the chemical composition of the polymer particles, by changing the lacquer or gel composition and/or by altering the interaction between these polymer portions and the lacquer or gel composition. However, the abrasion resistance remains an issue to be solved.

Therefore, development of abrasion resistance, hardness and flexibility for nail coatings constitutes at least some of the design parameters for modern nail coatings.

### SUMMARY OF THE INVENTION

These and other needs are achieved by the present invention which constitutes in one aspect embodiments of a composition of a stable dispersion of coated glass microparticles in a UV gel, hereinafter a particulate-gel composition. It has been found that application and curing of embodiments of this particulate-gel composition onto a substrate such as a nail plate delivers a cured nail coat containing coated glass microparticles that is smooth, has significant abrasion resistance and significant tensile strength. The microparticles constitute very small micron sized particles of silicon dioxide glass having a longest dimension ranging from sub-micron to about 45 microns (u). The stable dispersion of embodiments of this composition is believed to be achieved at least in part by an organosilane coating on the glass microparticles.

Embodiments of the UV gel components are directed to one or more unsaturated carboxylate compounds comprising a) one or more (meth)acrylate monomers and/or crotonate monomers alone, or b) one or more bis(meth)acrylate multimers and/or crotonate multimers alone, or c) a combination of monomers and multimers. The UV gel components also include at least one photoinitiator. UV gel additives and ancillary agents may also be included in the UV gel.

A further aspect of the invention concerns the relationship between the average dimension of the coated glass microparticles, the weight percentage of the portion of the coated glass microparticles combined with the UV gel and the count of number of particles combined with the UV gel as a function of their dimension and the weight percentage of portion of coated glass microparticles added.

Additional aspects of the present invention are directed to embodiments of methods for preparing the particulate-gel composition and embodiments for application of the particulate-gel composition onto a nail plate to form a composite nail coat and cure it to provide a cured nail coat. Practice of embodiments of the method using the particulate-gel composition to produce the composite nail coat and UV curing it to produce the cured nail coat on one or more nail plates delivers a cured nail coat exhibiting superior abrasion resistance, high tensile strength, a smooth surface and a bright appearance. A further aspect of the present invention is directed to an applicator kit constituting at least a container holding the particulate-gel composition.

According to embodiments of the method of application according to the invention, transformation to cure and/or convert the particulate-gel composition may be accomplished by UV light/actinic radiation. In practice, the transformation is accomplished by applying the particle-gel composition to a substrate to produce a composite nail coat in an uncured state on the substrate and exposing the composite nail coat to UV light/actinic radiation to cure the UV gel. Optional drying of the composite nail coat to evaporate optional non-reactive organic solvent and at least in part further solidify the composite nail coat may also be practiced along with optional mechanical smoothing of the composite nail coat surface. Upon UV curing, the resultant polymerized, cured gel constitutes a solid nail covering in which is embedded the dispersion of coated glass microparticles as particulates, hereinafter the cured nail coat.

Another aspect of the invention is directed to embodiments of a kit for practice of the method according to the invention. Embodiments of the kit include but are not limited to a unit container of the particulate-gel composition, an optional brush for application of the composition to a nail plate, an optional UV light irradiation apparatus and optional instructions for use.

A further aspect of the invention is directed to embodiments of the cured nail coat on a nail plate wherein the coat constitutes a UV cured composition of the primary components, the polymerized gel in which is embedded with coated glass microparticles. Embodiments of the cured nail coat present a contiguous film of polymerized gel with dispersed coated glass microparticles. The cured nail coat has high tensile strength, is significantly abrasion resistant and has a smooth, bright coating surface. Further embodiments of the cured nail coat include optional inclusion of ancillary agents such as but not limited to domains of preformed film formers and/or colorants and/or pigments.

### BRIEF DESCRIPTION OF THE FIGURES

FIG. 1 shows the separation of glass microparticles and Gel.

FIG. 2A shows the suspension of 1 micron glass microparticles in Gel.

FIG. 2B shows the suspension of 2 micron glass microparticles in Gel.

FIG. 2C shows the suspension of 10 micron glass microparticles in Gel.

FIG. 3 shows the substantial separation of 65 micron glass microparticles in Gel.

FIG. 4 shows the viscosity graph of several UV gels with and without glass microparticles.

FIG. 5A1 shows the pre-scrub view on black substrate of glass microparticles with Gel.

FIG. 5A2 shows the pre-scrub view on black substrate of Gel only.

FIG. 5B1 shows the post-scrub view on black substrate of glass microparticles with Gel.

FIG. 5B2 shows the post-scrub view on black substrate of Gel only.

### DEFINITIONS

Unless defined otherwise, all technical and scientific terms used herein have the same meaning as commonly understood by a person of ordinary skill in the art.

As used in the specification and the appended statements and claims, the singular forms “a,” “an” and “the” include plural referents unless the context clearly dictates otherwise. Also use of a plural term describing a thing or element includes the singular unless the context clearly dictates otherwise. For example, the term (meth)acrylates includes a single (meth)acrylate as well as multiples.

The term “may” in the context of this application means “is permitted to” or “is able to” and is a synonym for the terms “can” and “is.” The term “may” as used herein does not mean possibility or chance.

The term and/or in the context of this application means one or the other or both. For example, an aqueous solution of A and/or B means an aqueous solution of A alone, an aqueous solution of B alone and an aqueous solution of a combination of A and B. When more than two items are referred, the term and/or also means any combination of these multiple items as well as all and each.

The term “about” as used herein, when referring to a numerical value or range, allows for a degree of variability in the value or range, and is understood to mean±10%, of a stated value or of a stated limit of a range.

If a value of a variable that is necessarily an integer, e.g., the number of carbon atoms in an alkyl group or the number of substituents on a ring, is described as a range, e.g., 0-4, what is meant is that the value can be any integer between 0 and 4 inclusive, i.e., 0, 1, 2, 3, or 4. Similarly, values expressed in a range format should be interpreted in a flexible manner to include not only the numerical values explicitly recited as the limits of the range, but also to include all the individual numerical values or sub-ranges encompassed within that range as if each numerical value and sub-range were explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range.

The terms “comprising”, “comprises” and “comprised of” as used herein are synonymous with “composed of”, “including”, “includes” or “containing”, “contains”, and are inclusive or open-ended and do not exclude additional, non-recited members, elements or method steps.

For the purposes of the presently claimed invention, “% by weight’ or ‘wt. %” as used in the presently claimed invention is with respect to the total weight of the recited composition. Further, the sum of wt. % of all the compounds (components), as described herein, in the respective compositions add up to 100 wt. %. Accordingly, all percents of ingredients or components are given as weight-percentages relative to the total weight of the composition in which the ingredients and/or components are present, unless otherwise stated.

All average molecular weights of polymers are weight-average molecular weights, unless otherwise specified.

The term “substantially free” as the term is used herein means completely or almost completely; for example, a composition that is “substantially free” of a component either has none of the component or contains such a small amount that any relevant functional property of the composition is unaffected by the presence of the small amount of the component in question. A compound that is “substantially pure” has only negligible traces of impurities present.

The term “substantial” means a significant amount such as more than a majority amount. For example, a mixture of compounds A and B in which A is present in a substantial amount means that A is present at a weight percent or number of moles that is greater than the weight percent or number of moles of B. This term also means more than a minimal characteristic, examples of which include substantial flow or substantial transformation.

The terms “essential” and “essentially” are encompassed by the terms substantial and substantially so as to cover a smaller range than the terms substantial and substantially. The terms essential and essentially relating to purity or result may be regarded as equal to or more than 98% of the theoretic 100% purity or result.

The term longest average dimension means herein the average of the longest dimension of a three dimensional object such as but not limited to a polygon, a tetrahedron, a shard, a flake, a three dimensional trapezoid, a cube, a block or similar three dimensional object. The average may be determined by measuring the longest dimensions of a group of objects and averaging these measurements. For a large group of very small objects, the average may be determined by statistical analysis based upon sampling and the methods for production and separation such as by screen sizing.

The following groups of terms are used throughout this application: 1) preferred, preferably and preferable; 2) more preferred, more preferably and more preferable, 3) especially more preferred, especially more preferably and especially more preferable; 4) most preferred, most preferably and most preferably; 5) especially most preferred, especially most preferably and especially most preferable; and 6) very especially most preferred, very especially most preferably and very especially most preferable. These groups convey a meaning of preference for a group of substituents, structures, moieties, components and compounds. The degree of preference is self-explanatory by the terms themselves. Within each group, the meanings of the synonyms, preferred, preferably and preferable are the same. There is no difference in meaning in the context of this application when a group is described in a particular sentence as preferred and then in another sentence this same group is described as preferably. Not all six categories of preference are used in this application to describe each and every substituent, formula, subgenus integer symbol and atom designator. In some instances, two or three categories are used while in other categories five or six categories are used. The degree of preference as expressed by these terms for members of series which progress from many to a few individually named components is self-explanatory and internally consistent for the particular series being described. Reference throughout this specification to “embodiment”, “one embodiment” or “preferred embodiment” or “more preferred embodiment” or “most preferred embodiment” means a feature of any one or more elements and can be used in connect with different elements of the invention.

The term film former means a fully formed polymer such as a poly(meth)acrylate, polyester, polyamide, polyurethane, cellulosic ether or ester or shellac that will form a film or layer when the film former in an organic or aqueous medium is coated on a substrate and dried to remove the medium. The film former typically does not undergo substantial cross-linking or other chemical reaction after its deposition as a film.

The term (meth)acrylate means either one alone and/or both of an acrylate ester and a methacrylate ester. When an alkyl methacrylate ester alone is to be described, the parenthesis around “meth” is excluded and the resulting term means the 1-methyl-1-carbanoyloxyalkyl ethene or alkyl methacrylate. In a similar fashion, the term alkyl acrylate means 1-carbanoyloxyalkyl ethene or alkyl acrylate.

The term surfactant means a zwitterionic, nonionic, anionic or cationic compound having lipophilic and hydrophilic qualities so that it can function to solubilize lipophilic substances in hydrophilic and/or aqueous media. A surfactant may also perform as a plasticizer in polymer compositions to provide flexibility to the polymer composition.

The term plasticizer means a compound that may be soluble to dispersible in a solid material such as a polymer and enables the macromolecular configuration of the mixture to exhibit flexibility not present in the polymer alone often by lowering the glass transition temperature curve of the polymer. Plasticizers for polyolefins and functionalized polyolefins are well-known. Examples include alkyl phthalate esters, alkyl adipate esters, alkyl sebacate esters, glycerol triacetate (triacetin) and acetyl tributyl citrate.

The term rheologic control agent means a rheology modifier that will thicken an otherwise free-flowing liquid composition. The modification renders the composition flowable when applied by spray, brush or other coating method but the composition will remain in a static position in a quiescent state. These agents are typically classed as thixotropic agents. Examples include polyvinyl alcohol, a polyethylene glycol, vegetable gum, laponites, and hydrocarbon wax.

The term alcohol means a mono, di, tri or polyol that can solubilize other components of the composition. Alcohols include methanol, ethanol, propanol, butanol, ethylene glycol (ethylene diol), glycerin (glycerol), propylene diol, or di-ethylene glycol, di-propylene glycol as well as glyme (methoxylated ethylene glycol). Preferred alcohols include but are not limited to methanol, ethanol, propanol, ethylene glycol, propylene glycol and glycerin.

The term gel means a liquid within a three-dimensional network that forms at least a semi-solid-like consistency in the static, quiescent state. The gel has sufficient rheologic control and/or density and/or viscosity at rest (static state) to maintain continuous integrity of the liquid gel as a coating or layer on a flat or curved surface. The gel character of the liquid means that the liquid will not spontaneously flow off a surface on which it has been coated but can be readily removed by mechanical force such as by wiping with a cloth or tissue.

The term flowable means a liquid that will flow like water or an aqueous latex paint when contacted by mechanical means such as by a brush, sponge or other applicator.

Together, the terms gel and flowable mean that the liquid having these characteristics is thixotropic.

The term alkylenyl used in the present invention means a linear saturated hydrocarbon chain with open valences at both termini. An example is hexylenyl of the formula —(CH₂)₆—.

The term nail plate means a human fingernail, a human toenail as well as an artificial nail extension.

The term “user” means the person preparing and applying a nail coating composition such as the particulate-gel composition of the present invention. The user may be, for example, a professional manicurist working in a salon who is different from the subject on whose nails the composition is to be applied. The user, for example, may also be identical to the person on whose nails the composition is applied.

In the following passages, different aspects of the subject matter are defined in more detail. Each aspect so defined may be combined with any other aspect or aspects unless clearly indicated to the contrary. In particular, any feature indicated as being preferred or advantageous may be combined with any other feature or features indicated as being preferred or advantageous. A particular feature, structure, or characteristic described in connection with an embodiment may not be the same as another feature, structure or characteristic of another embodiment of the presently claimed invention. Thus, appearances of the phrases “in one embodiment” or “an embodiment” or “in a preferred embodiment” in various places throughout this specification are not necessarily all referring to the same embodiment but may refer to different embodiments of the presently claimed invention. Furthermore, the features, structures, or characteristics in one or more embodiments may be combined in any suitable manner, as would be apparent to a person skilled in the art from this disclosure. Furthermore, while some embodiments described herein include some, but not other features included in other embodiments, combinations of features of different embodiments are meant to be within the scope of the subject matter, and form different embodiments, as would be understood by those skilled in the art. For example, in the appended claims, any of the claimed embodiments can be used in any combination.

Reference throughout this specification to “one embodiment” or “preferred embodiment” means that a particular feature, structure, or characteristic described in connection with the embodiment is included in at least one embodiment of the presently claimed invention. Thus, appearances of the phrases “in one embodiment” or “in a preferred embodiment” in various places throughout this specification are not necessarily all referring to the same embodiment but may refer to different embodiments of the presently claimed invention. Furthermore, the features, structures or characteristics may be combined in any suitable manner, as would be apparent to a person skilled in the art from this disclosure, in one or more embodiments. Furthermore, while some embodiments described herein include some, but not other features included in other embodiments, combinations of features of different embodiments are meant to be within the scope of the subject matter, and form different embodiments, as would be understood by those in the art. For example, in the appended claims, any of the claimed embodiments can be used in any combination.

### DETAILED DESCRIPTION OF THE INVENTION

Embodiments of the present invention deliver a cured nail coat on a nail plate having superior abrasion resistance, high tensile strength, and a smooth, shiny surface. Embodiments of the cured nail coat comprise embedment in a cured UV gel coat of coated glass microparticles having an average dimension of submicron size, e.g., 0.001-0.01 microns, up to about 45 microns. The cured nail coat is formed by actinic curing of embodiments of a Particulate-Gel Composition which is a stable dispersion of coated glass microparticles in the UV gel. The cured nail coat is tough, strong and has high tensile strength, superior abrasion resistance and a smooth surface with desirable sheen and brightness.

### The Particulate-Gel Composition

According to embodiments of the Particulate-Gel Composition of the invention, coated glass microparticles are substantially to essentially uniformly dispersed in the UV gel to form a stable dispersion. Embodiments of the Particulate-Gel Composition may be subsequently applied to a nail plate and polymerized by UV curing with optional drying to form on the nail plate a high tensile strength, abrasion resistant coat or covering of a cured UV monomer/multimer film with essentially uniformly dispersed coated glass microparticulates.

The stable dispersion of embodiments of the Particulate-Gel Composition is believed to be achieved at least in part by a tendril-like structure of the coating on the glass microparticles. The tendril head of the coating is believed to interact in some fashion with the silicon dioxide of the microparticles and the tendril tail of the coating is believed to interact in some fashion with the UV gel. The stability is demonstrated by the ability of the dispersion to be maintained for long periods of time ranging up to months to years. This aspect of the dispersion according to present invention enables embodiments of the particulate-gel composition to be stable and storable. These embodiments provide a single unit bottle product to be applied as a ready-to-use nail coating composition.

### The Coated Glass Microparticles

The coated glass microparticles constitute micro-particles of silicon dioxide glass having a longest dimension ranging from sub-micron to about 45 microns (u), preferably ranging from about 0.01 micron to about 30 microns, more preferably ranging from about 0.1 micron to about 20 microns with a statistical deviation of about +10-30%, especially more preferable ranging from 0.1 micron to about 10 microns with a statistical deviation of about +10-30%.

The use of a submicron to micron diameter range of the glass microparticles is enabled at least in part by the coating on the microparticles which establishes formation of a long lasting stable dispersion of the microparticles in the UV gel as described above. Without the coating, microparticles of submicron and small micron size tend to form a non-uniform mixture of agglomerates of microparticles in the UV gel. Larger micron sized microparticles with no coating tend to form unstable mixtures in the UV gel. All submicron and micron sized microparticles with no coating tend to separate from the UV gel into settled microparticles and UV gel mixed with little or no microparticles. These dispersions of uncoated microparticles typically become unstable and separate within hours to days after formation.

The formation of the stable dispersion enables use of a range of micron and submicron sizes because the stable dispersion prevents the clumping together of smaller micron sized microparticles. The stable dispersion also prevents untoward settling of the higher micron sized microparticles. The interaction of the coating tendrils with the UV gel and the at least moderate viscosity of the UV gel enables a substantially to essentially stable dispersion of the coated glass microparticles. The stable dispersion also enables a substantially to essentially uniform distribution of all sizes of the microparticles in the gel and in the cured coat.

### Glass Microparticles

The glass microparticles are constructed of silicon dioxide glass such as soda lime silicate or borosilicate or barium silicate glass. Silica, silicate and silicon dioxide are synonyms for the amorphous SiO₂ polymer constituting the chemical constitution of the glass microparticles. The glass microparticles are commercially available from such sources as Healthcare Products, Inc of San Diego California. The glass microparticles may have regular and/or irregular shapes. Regular shaped glass microparticles include but are not limited to regular configurations such as spherical, elliptical or egg shaped, rods, needles, flakes, cubes, boxes and any configuration wherein all or substantially all of the glass microparticles have the same shape and the shape has repeating lines and/or curves resulting from comminuting silicate in a molten or soft form or other malleable form to produce repeating regular shapes of microparticles. The irregular shaped microparticles may be any non-repetitious configuration and/or combination of irregular configurations including but not limited to irregular flakes, shards, irregular needles, rocks, irregular prisms, irregular blades, polygons, tetrahedrons, irregular cubes, irregular boxes, blocks, irregular spheres, irregular egg-shapes and/or any irregular shape that may result from shattering, grinding, pressing, crushing, smashing, pulverizing and/or otherwise breaking and/or comminuting large pieces of silicon dioxide glass such as but not limited to glass blocks or rock-like pieces measuring at least centimeters on a side.

The glass microparticles have an average dimension measured as their longest average irregular dimension and/or longest average regular dimension ranging from submicron of micron sizes as described above and typically constitute a range of dimensional sizes distributed according to a Gaussian curve having the average dimension as its maximum. Preferably, the Gaussian distribution of microparticle sizes having the average longest dimension at its maximum may be 30% more preferably 50%, especially more preferably 70%, at the half height point of the Gaussian curve. Alternatively, the dimensional size deviation depends upon the average dimension and the sizing technique used. The size deviation may range ±˜0.1μ to ±˜30μ depending upon the longest dimension of the maximum number of glass microparticles of the group of variable dimension microparticles to be used.

### Concentration and Number of Coated Glass Microparticles for the Particulate-Gel Composition

Embodiments of the Particulate-Gel composition incorporate a concentration of the dispersed, coated glass microparticles in the microparticle-gel composition in a weight percentage relative to the total weight of the particulate-gel composition. This weight percentage of coated glass micropoarticles may range of from about 1 wt % to about 60 wt %, preferably about 2 wt % to about 50 wt %, more preferably about 10 wt % to about 40 wt % and especially more preferably about 10 wt % to about 35 wt %. As explained below, the selection of weight percent depends upon the dimensional size of the coated microparticles, the viscosity of the UV gel, and the suspension quotient of the organosilane coating. Use of significantly higher dimensional sizes generally lead to selection of higher weight percentages of coated microparticles relative to significantly lower dimensional sizes. Accordingly, the weight percentage of the coated glass microparticles relative to the weight of the UV gel alone will be proportionately higher. For example, at 30 wt % of coated glass microparticles in 10 gm of particulate-gel composition, the 3 gm portion of coated glass microparticles and its corresponding 7 gm portion of UV gel to make up the 10 gm of particulate-gel composition. Relative to this amount of UV gel alone, the weight percentage of added coated glass microparticles=3/7 or about 43 wt %. relative to the UV gel alone.

These same weight percentages preferably adjusted according to the microparticulate number evaluation discussed below will provide effective cured particulate-gel coats on substrates such as a nail plate.

As the size or dimension of the coated glass microparticles increases, the number of microparticles present in a fixed weight of a portion of microparticles to be added decreases. Regular and/or irregular shape is also a factor for inter-relation between microparticles and the UV gel in the uncured and cured state. Because microparticle numbers present in the UV gel relate to development of the desirable properties of the particulate-gel composition in cured and uncured states, these factors have a bearing on the weight of coated microparticles to be added to the UV gel. Generally, as the dimensional size of the coated microparticles increases, the weight percentage of the coated microparticles to be combined with the UV gel will be increased so as to maintain a desirable level of the microparticle number average range. The weight increase factor ranges from negligible for slight changes in the dimensional size to as much as 75% or more, preferably 50% or more for significant changes in dimensional size relative to the weight of the lowest dimensional size of coated microparticles to be combined with the UV gel according to the invention.

More specifically, the relationship between the average dimensional microparticle size and number of microparticles relates to the number of microparticles added to a fixed quantity of UV gel. This number affects the properties of the cured and uncured particulate-gel composition. It is clear that a cured particulate-gel composition containing only two or three coated glass microparticles per cc would not have physical properties the same as a cured particulate-gel composition containing millions of coated glass microparticles per cc. Consequently, the number of coated glass microparticles present in a particulate-gel composition has a bearing on the properties of the particulate-gel composition in its uncured and cured states.

The coated glass microparticulate number relates to different glass microparticle sizes, glass microparticle shapes and the ability or inability of the glass microparticles to close-pack, i.e., form an arrangement in which the surfaces of the glass microparticles contact each other with no space therebetween. As the glass microparticle size becomes larger and/or the shape becomes more irregular, the number of glass microparticles in a fixed weight of coated glass microparticles decreases. Generally, at the limits of the coated glass microparticle average dimension range, a fixed weight of the 0.01 micron microparticles will contain significantly more particles that will the same fixed weight of the 45 micron coated glass microparticles. If the coated glass microparticles were spherical, the relationship might be considered to be somewhat linear. However, because the coated glass microparticles typically are a variation of shapes, a fixed weight of different batches of coated glass microparticles having the same average dimension may but not necessarily provide somewhat different microparticle number counts. Also, the Gaussian distribution discussed above will affect the number of coated glass microparticles in fixed weights from batch to batch.

This relationship between average size and number can be evaluated with idealized glass microparticle cubes. Consider a cc of borosilicate glass as a single cube and as a multitude of close-packed cubes measuring certain microns per side (e.g., regular shaped glass microparticles). At a typical weight of borosilicate glass of 2.51 gm per cc, 2.51 gm or 1 cc of idealized microparticle cubes of 1 micron per side will contain 10¹² microparticles in 1 cc (1 cm=10⁴μ: 10⁴×10⁴×10⁴=10¹²). Similarly 2.51 gm of idealized microparticle cubes of 80 microns per side will contain 1.953×10⁶ particles: (each side is 80 times larger than the cube with 1μ sides so that in 10⁴μ or 1 cm there are 80 times fewer cube sides or 1/80×10⁴ sides. Cubing these sides yields (0.0125×10⁴)³=(1.25×10²)³ or 1.953×10⁶ particles) In other words, 2.51 gm of 1 micron cube microparticles will contain about 0.5×10⁶ more particles or one half million more particles than the same weight of 80 micron cube microparticles.

As explained above, the particle number makes a difference for the behavior of the cured particulate-gel product. At a 25 wt % loading of ideal cubed microparticles per 10 gm of UV gel or 2.5 gm, the 1 micron cubed microparticles (10¹² microparticles) deliver 10³ or about 1000 more particles than does the same weight of 10 micron cubed microparticles [(10³)³=10⁹ microparticles]. Ideally, to provide the same particle number, about 2.5×10⁻⁶ gm or 2.5×10⁻³ mg extra weight of the 10μ microparticles are needed to add 10³ more particles.

These idealized cubed regular shape microparticles do not represent the actual coated glass microparticles. Real features including the irregular and/or regular shapes, a Gaussian size distribution of regular and/or irregular shapes, loose particulate packing and the dynamic interaction of the coating will be factors. The irregular and/or regular shape packing, the dynamic coating interaction, Gaussian distribution and the presence of the UV gel will create, expand and fill space between the coated glass microparticles. A rough evaluation of the spacing for coated glass microparticle numbers per cc in the UV gel may be determined by the weight percentage of coated microparticles added to the particulate-gel composition. For example, at 30 wt %, the one cc of the combination contains 0.3 gm of coated microparticles, assuming that the total weight of the cc of the particulate-gel composition 1 gm (density of HEMA is 1.073 g/ml). This lower weight of coated microparticles factors into the particle numbers present by 0.3/2.51=a multiplication factor of 0.12 for the microparticle numbers. If it is assumed that the space between coated microparticles created by the coating interaction is at least the same volume as the coated microparticles themselves, the space factor halves again the microparticle number. Together these factors accounting for the mix of coated microparticles in UV gel yield a total multiplication factor of 0.12×0.5=0.06. For the 1μ coated microparticles the UV gel factor provides a particle number of 10¹²×0.06 or 6×10¹⁰ particle numbers.

A practical evaluation of the UV gel factor for larger sized coated microparticles accounts for increased UV gel space between these coated microparticles because of their larger shapes irrespective of whether the shapes are regular or irregular. It is reasonable to assess this larger shape factor according to the increase in average size. Hence, an average coated microparticle size of 10μ will have a 10× greater UV gel space than will the 1μ size coated microparticles. The ideal microparticle number for the 10μ microparticle cube described above is 10⁹ microparticles. Adding the UV gel space factor of 0.06 and the increased UV gel space of 10× because of microparticle size, the coated microparticle number for the 10μ size at 30 wt % yields 0.006×10⁹ or 6×10⁶ microparticles. Compared with the 1μ coated microparticle number calculation above, particulate-gel composition with the 10μ coated microparticles contains 10⁴ fewer coated microparticles than does the particulate-gel composition with same weight percentage of 1μ coated microparticles.

Accordingly, these factors and the need to provide a particulate number range in the neighborhood of 10⁸ to 10¹² microparticles combine to provide a weight percentage multiplication factor of from about 1% to 40%, preferably about 2% to about 30%, more preferably about 5% to about 25% to be added to the selected weight percentage of the higher average size coated glass microparticles relative to the same selected weight percentage of 1μ coated microparticles.

### The Coating

The coating of the glass microparticles according to the invention comprises a head-tail compound of molecular weight no more than about 1500, preferably no more than about 1000, more preferably no more than 800. The head-tail compound has a core substituted by groups that are capable of interacting by chemical bonding and/or non-bonding interaction with the silicon dioxide of the glass microparticles and the compounds of the UV gel by dynamic exchange such as but not limited to electrostatic interaction, hydrogen pseudo bonding, polar interaction, lipophilic interaction and/or molecular-mechanical entwinning.

The core of the head-tail compound may be organic (carbon) or silicon based and may be substituted at its head by interaction groups such as hydroxyl and/or alkoxy groups and at its tail by an amine or mercaptan or (meth)acrylate or crotonate groups. Exemplary compounds are head-tail compounds such as but not limited to:
a) (mono, di or tri-hydroxyalkylenyl)alkyl amine,
b) (mono, di or tri-hydroxyalkylenyl)alkyl mercaptan,
c) hydroxyalkylenyl (meth)acrylate,
d) (mono, di or tri-hydroxyalkyenyl)siloxy alkylenyl (meth)acrylate,
e) (mono, di or tri-alkoxy)siloxy alkyenyl (meth)acrylate and/or
f) The crotonate analog of (meth)acrylate compounds of c), d) and/or e).
   The alkylenyl and alkoxy moiety of exemplary compounds a)-f) may be from 1 to 4 carbons, preferably from 1 to 3 carbons.

Preferred head-tail compounds are the siloxy alkylenyl (meth)acrylate compounds wherein siloxy is substituted by two or three hydroxyalkyenyl or two or three alkoxy groups. More preferred is trimethoxysiloxy methylenyl or ethylenyl or propylenyl (meth)acrylate and triethoxysiloxy methylenyl or ethylenyl or propylenyl (meth)acrylate with the acrylate version of (meth)acrylate being more preferred.

A more preferred alkoxysiloxy alkylenyl (meth)acrylate is the siloxy compound of Formula I.

(R¹O)ₙ(R²)₃₋ₙSiO—(R²₂SiO)ₐ—R²SiO—R³—O₂CC(R⁴)═CH₂ Formula I

An especially more preferred alkoxysiloxy (meth)acrylate is the siloxy compound of Formula II.

(R¹O)ₙ(R²)₃₋ₙSiO—R³—O₂CC(R⁴)═CH₂ Formula II

For Formulas I and II, R¹ is methyl or ethyl, R² is methyl, R³ is methylenyl, ethylenyl, propylenyl, R⁴ is H or methyl, the designator a is zero or an integer of 1 to 5 and the designator n is an integer of 1, 2 or 3.

A most preferred alkoxysiloxy alkylenyl (meth)acrylate is Formula II wherein n is 3, R¹ is methyl, R³ is ethylenyl or propylenyl and R⁴ is H.

It is believed that the head-tail compound may be capable of chemical bonding or non-bonding interaction of its head of hydroxylalkyl or alkoxy groups with the silicon dioxide of the glass microparticles. The alkyl (meth)acrylate, alkylamine or alkylmercaptan tail of head-tail compound is believed to behave in a tendril-like fashion extending from the microparticles so that the tendrils may engage in non-bonding interaction with the (meth)acrylate compounds of the UV gel.

Exemplary glass microparticles coated with a preferred coating compound, such as the triethoxysiloxypropyl (meth)acrylate, may also be obtained from commercial sources such as Healthcare Products Inc. or may be prepared according to the foregoing process using re-crushed glass shards from companies such as Jeejunye, Etsy, American Specialty Glass and others.

### Coating Formation on Microparticles

A coating compound such as triethoxy- or trimethoxy-(meth)acryloxypropyl silane (a.k.a triethoxy or trimethoxy siloxy propylenyl (meth)acrylate herein) or a trimethoxy or triethoxy alkyl compound having an amine, mercaptan or (meth)acrylic tail may be combined with the glass microparticles by hydrolytic reaction of the coating compound and the silicon dioxide of the glass microparticles. A mixture of uncoated glass microparticles in an aqueous or aqueous-organic medium may be prepared and combined with the coating compound to form a coatable mixture. The coatable mixture may be homogenized in an agitator mill or similar device until the coating compound has become interactively combined with the glass microparticles. Excess coating compound and medium may be separated from the coated glass microparticles by filtration and the coated glass microparticles dried by vacuum evaporation. See for example U.S. Pat. No. 8,349,399, assignee Schott AG, issued Jan. 8, 2013, the disclosure of which is incorporated herein in its entirety.

The amount of coating compound used for coating the glass microparticles may range from about 0.1 wt % to about 30 wt %, preferably from about 0.5 wt % to about 20 wt %, more preferably from about 0.5 wt % to about 15 wt % relative to the weight of the glass microparticles. Within these ranges, the amount of coating compound to be used increases with decreasing average longest dimension of the glass microparticles. A smaller average longest dimension means higher surface area and consequently more coating compound is needed to cover the higher surface area.

The process delivers a substantially uniform coating of the coating compound on the surfaces of the glass microparticles. The amount of coating compound used to coat will not in most situations equal the amount of coating compound covering the glass microparticles but instead will be higher. The amount used is sufficient to provide the substantially uniform contiguous coating. The excess coating compound and medium may be removed by filtration and the coated glass microparticles may be subsequently dried. It is believed that the coating comprises at least a contiguous film on all or essentially all surfaces of the glass microparticles. The film thickness and its attendant tendrils are sufficient to provide the dispersion stability of the particulate-gel composition. The non-bonding interaction of the coating tendrils and the UV gel is believed to be sufficient to enable the stable dispersion of the glass microparticles in the UV gel according to the weight percentages described above. See above cited '399 patent for a description of the process for coating small particles such as but not limited to glass microparticles.

The balance between the stability promotion associated with the trialkoxy silyl alkyl acrylate interaction with the UV gel is dependent upon the weight average weight of the glass microparticles. The viscosity of the UV gel and the length of the alkylacrylate tendrils combine to provide the weight supporting interaction of the glass microparticles and provide the stability of the dispersion against settling upon storage. However, when the weight average weight of the glass microparticles becomes larger than about 45 microns, the weight average weight tends to overcome the weight supporting interaction. While the settling at larger weight average weights above 45 microns is not immediately apparent upon mixing, over a period of time such as hours to days, settling may occur. It has been found, however, that with weight average weights up to about 45 microns, the weight supporting interaction enables maintenance of a stable dispersion for months.

Surprisingly, the length of the alkylacrylate tendrils actually affects dispersion stability. While it may be expected that longer alkylacrylate lengths would contribute to better tendril entangling, beyond an alkyl length of about six carbons, e.g., hexyl, the glass microparticles tend to sag and/or experience greater translational motion in the UV gel. These effects tend to enable slow settling of the dispersion.

### The UV Gel

The UV gel comprises at least one or more unsaturated carboxylate compounds. The one or more unsaturated carboxylate compounds comprise one or more (meth)acrylate and/or crotonate monomer, preferably a (meth)acrylate monomer alone. Alternatively, the one or more unsaturated carboxylate compounds comprise at least one (meth)acrylate and/or crotonate multimer, preferably a (meth)acrylate multimer alone. Alternatively, the one or more unsaturated carboxylate compounds comprise a combination of the one or more monomers and one or more multimers. The UV gel also comprises at least one photoinitiator.

Embodiments of the UV gel have viscosities ranging from about 2K (2000) cP to about 500K cP, preferably from about 2K to 450K cP, more preferably about 2K to about 400K cP. Exemplary viscosities may be 1K to 3K cP, 2K to 4 K cP, 5K to 10K cP, 7K to 15K, 350K to 400K cP. The UV gel usually does not have thixotropic character; however, additives may confer thixotropy to the UV gel.

### Monomer or Multimer or Combination Thereof

The UV gel may comprise one or more monomers alone, one or more multimers alone or a combination of one or more monomers and one or more multimers. When the UV gel comprises solid monomers alone or solid multimers alone, the UV gel optionally comprises solvent to provide a flowable gel. Alternatively, one or more monomers that are liquid at STP may be included in UV gel of one or more monomers alone to provide liquidity. Alternatively, one or more multimers that at liquid at STP may be included in the UV gel of one or more multimers alone to provide liquidity.

The monomer compound comprises a C1-C20 alkyl and/or C2-C20 hydroxyalkyl and/or a C5-C16 cycloalkyl (meth)acrylate ester or crotonate ester monomer. Preferably the alkyl, hydroxyalkyl and cycloalkyl carbon number are respectively C1-C15, C2-C15 and C5-C14, more preferably C1-C10, C2-C10, C5-C12 respectively and especially more preferably C1-C6, C2-C6 and C5-C10 respectively. Preferably, the monomer may be methyl and/or ethyl and/or hydroxymethyl and/or hydroxyethyl (meth)acrylate and/or hydroxypropyl (meth)acrylate, more preferably methyl and/or ethyl and/or hydroxyethyl and/or hydroxypropyl acrylate. Additional liquid (meth)acrylate monomers such as isobornyl (meth)acrylate, cyclohexyl (meth)acrylate may be included to provide semi-liquidity to the gel, especially as the gel cures.

The multimer compound comprises a (meth)acrylate or crotonate multimer with a urethane, ester, amide, polyol or dimethyl siloxane multi chain oligomeric link between the multiple (meth)acrylate or crotonate groups terminating multiple chains of the multimer. Preferably, the multimer may be a tetramer having four terminal (meth)acrylate or crotonate groups, a trimer having three terminal (meth)acrylate or crotonate groups and/or a dimer having two terminal (meth)acrylate or crotonate groups or any combination thereof. Preferably, the multimer is a tetramer, trimer or dimer or a combination thereof. More preferably, the multimer is a major portion of dimer and a minor portion of a trimer. Alternatively, the multimer comprises the dimer alone. The multimer average mw's may range from about 150 Da to about 750 KDa, preferably about 150 Da to 250 KDa. Mixtures of multimers may comprise from 50 wt % to 75 wt % of average mw of 150 Da to 2 KDa and a remainder of average mw of 10 KDa to 750 KDa of multimer relative to the total weight of multimer. Preferably, the major portion of multimer will be a dimer with the minor portion being trimer, tetramer or mixture thereof.

The multimer with the oligomer of urethan, ester or dimethyl siloxane is formed by combination of (meth)acrylic or crotonic acid with the corresponding multi-chain oligomer link coupler. If the corresponding oligomer link is a urethane and the termini are residues of (meth)acrylic or crotonic acid, the coupler may be a multi-isocyanate terminated oligo-urethane produced from a C3-C10 alkane diisocyanate and a C2-C8 alkanediol, triol or tetraol, hereinafter a multi-isocyanato multiunit alkyl urethane oligomer. Esterification of the multi-isocyanato groups of the oligomer with a hydroxyalkyl (meth)acrylate or crotonate ester provides the multimer. The oligomer may have multiple isocyanate terminating groups for a tetramer, a trimer and/or a dimer; preferably a trimer and/or a dimer, more preferably a dimer. With a similar esterification step, if the corresponding oligomeric link is an ester, the coupler may be a multi-carboxy terminated oligo-ester produced from a C3-C10 alkanedioic acid and a C2-C8 alkanediol, triol and/or tetraol, hereinafter a multi-carboxy multiunit alkyl ester oligomer. If the corresponding oligomeric link is a polydimethyl siloxane, the coupler may be a multi-hydroxy multiunit polydimethylsiloxanyl oligomer. An alternative method of dimer formation may be practiced through use of a (meth)acrylic or crotonic acid and reaction of the hydroxy groups of the corresponding multi-hydroxyalkyl urethane, ester or polysiloxane coupler.

The multimer with an oligomer of an amide, polyol or alkylenyl is formed by combination of a (meth)acrylic acid or crotonic acid with the corresponding oligomeric coupler through an amidation or esterification. If the corresponding oligomeric link is an amide, the coupler may be a multi-amine multiunit amide oligomer produced from a C3-C8 alkanedioic acid and a C2-C8 alkane multi-amine. If the corresponding oligomeric link is a polyol, the coupler may be a multi-hydroxy polyol produced from a C2-C5 epoxy-alkane, e.g., ethylene oxide, propylene oxide, oxetane, tetrahydrofuran or oxane or may be a tri-, tetra- or penta-methylol alkane. If the corresponding oligomeric link is an alkylenyl, the coupler may be a multi-hydroxy C3-C20 alkane.

Preferred examples include but are not limited to an oligomer coupler at least di- or tri-terminated by a (meth)acrylate group wherein the oligomer coupler is:
a) a C4-C16 alkane diol (use (meth)acrylic acid), or
b) a 1-5 unit ethylene oxide oligomer (use (meth)acrylic acid), or
c) a 1-5 unit propylene oxide oligomer (use (meth)acrylic acid), or
d) trimethylolpropane (triterminated) (use (meth)acrylic acid), or
e) an α,ω-diisocyanoto (diterminated or α,ω,ω tri-isocyanato (triterminated) 1-5 unit C3-C9 alkyl urethane oligomer, or
f) an α,ω-dihydroxy (diterminated) or α,ω,ω tri-hydroxy (triterminated) 1-5 unit dimethylsiloxanyl oligomer (use (hydroxyalkyl (meth)acrylate).

More preferred oligomer multimer examples include b), c), d) and e) above as dimers. An especially, more preferred oligomer multimer example includes one or a combination of the dimers: α,ω-diisocyanato 1 to 3 unit C6-C9 alkyl urethane oligomer and an α,ω-diisocyanato 9 to 15 unit C6-C9 alkyl urethane oligomer. More preferably, the dimer may be α,ω-diisocyanato 2 or 3 unit C6-C9 alkyl urethane oligomer carbamated with a hydroxyalkyl (meth)acrylate or a di or tri ethylene oxide diesterified with acrylate.

When the UV gel comprises multimer alone, liquid diacrylates such as urethane di(meth)acrylate, glycol di(meth)acrylate, dimethyl siloxanyl di(methy)acrylate wherein the urethan, glycol or dimethylsilxoanyl unit contains from 1 to 5, preferably from 1 to 4 units. Examples of urethane dimers include Sinomer UVU6212, Aohui CR91638, IGM Resin 6210, Esstech PL-7003.

The UV gel alternatively may comprise a combination of one or more monomers and one or more multimers as described above. The monomer and multimer concentrations may be arranged so as to provide low to moderate to high cross linking, preferably high cross linking. A higher concentration of the multimer relative to the monomer will deliver higher crosslinking. A higher crosslinking result will contribute to a harder solid coating. The molar ratio of monomer to multimer wherein a mol of a multimer with multiple unsaturations is counted as one mol may range from about 20:1 to about 2:1, preferably about 10:1 to about 4:1, more preferably about 10:1 to about 3:1.

The total concentration of monomer alone or multimer alone or the combination of monomer and multimer may range from about 70 wt % to about 99% wt percent relative to the total weight of the UV gel composition (i.e., without coated glass microparticles), preferably from about 80 wt % to about 95 wt %, more preferably from about 95 wt % to about 99 wt % with the remainer being photoinitiator, and optionally solvent, if included, optional one or more ancillary agents and/or pigments and/or colorants.

A photoinitiator such as benzophenone, phosphine oxide, TPO, TPO-L, 1-hydroxycyclohexyl phenyl ketone, and similar type I and type II photoinitiators may be combined with the UV monomer/dimer gel to enable photopolymerization. The concentration of photoinitiator may range from 0.01 wt % to about 5 wt %, preferably 0.01 wt % to about 4 wt %, more preferably about 0.01 wt % to about 3 wt % relative to the total weight of the UV gel composition.

Photolysis of the particulate-gel composition with actinic radiation at wavelengths appropriate for the photoinitiator present converts the UV monomer/dimer to the solid, cured coat or film containing embedded coated glass microparticles as particulates.

Exemplary UV gels and exemplary compositional disclosures of suitable UV gels for use according to the present invention are described in U.S. Pat. Nos. 11,166,901 and 11,4395,573, the disclosures of which are incorporated herein by reference.

### Ancillary Agent

Incorporation of additives and ancillary agents in the UV gel may facilitate at least in part the properties of the particulate-gel composition and the cured nail coat. The additives and ancillary agents may be incorporated into the UV gel before or after its combination with the coated glass microparticles. Appropriate ancillary agents for the UV gel include but are not limited to one or more ancillary agents to provide rheology control, solubilization of individual components that are insoluble in each other, free radical scavenging, polymerization inhibition, thixotropy, plasticization, gel spreading and/or color provided that they do not chemically interact with the monomer and dimer, one or more of each of a non-reactive organic solvent, a free radical scavenger, a UV inhibitor, a surfactant, an oxygen scavenger, a thixotropic agent, a leveling and spreading agent, a plasticizer, an additional supplemental particulate, a preformed film former, a pigment, a colorant, and a reactive agent or an unsaturated monomer acting as a diluent as well as non-reactive volatile organic solvent. Optional ancillary nail coating materials may also be included such as but not limited to those that may deliver color, thixotropic properties, thickening properties, leveling properties, flow properties and flexibility properties to the uncured UV gel and/or its cured and/or polymerized and/or solidified state.

Ancillary agents providing at least in part these features include but are not limited to solvents, soluble and/or particulate pigments/colorants, gums such as locust gum, guar gum, acacia gum, xanthan gum and similar natural thickening agents; cationic polymers such as trimethylammonium alkyl cellulose, poly(trimethyl ammonium propyl (meth)acrylate); particulates such as silica, mordenite and/or bentonite and/or hectorite clay; one or more anionic and/or cationic and/or nonionic surfactants such as but not limited to lauryl sulfonate, laureth sulfonate, lauryl carboxylate, trimethylammonium halides of fatty C10-C26 alkyl/alkenyl groups such as behenyl, cetyl, stearyl, oleyl, myristyl or palmitoleyl; one or more plasticizers such as a phthalate ester, a trimellitate ester, an adipate ester or a dialkyl oligoglycol, or sucrose benzoate or trimethylpentanyl diisobutyrate or any combination thereof; a polysiloxane such as dimethicone or a polysilicone copolymer with ethylene oxide and/or propylene oxide oligomer or polymer block units; hydroquinone, 4-methoxylphenol, oxygen scavenger, adhesion promoting primers such as methacrylic acid, ethyl acetate, and other common additives for UV gels.

A preformed film former (PFF) may be optionally included with the UV gel to enable at least in part a lacquer-like shine. Additionally, a PFF having physical characteristics making it capable of forming a two phase solid or PFF domains with a continuous phase of the cured UV gel enable at least in part an easy, mild removal by organic solvents such as acetone, methyl ethyl ketone and similar solvents.

The PFF comprises a preformed non-reactive film forming polymer such as but not limited to nitrocellulose, ethyl cellulose, cellulose acetate, cellulose acetate butyrate, tosylamide epoxy resin, acrylates copolymer, a polyester such as adipic acid/neopentylglycol/trimellitic anhydride copolymer and/or a styrene/acrylates copolymer and any mixture thereof. Preferably, the PFF is one or more of nitrocellulose, ethyl cellulose, cellulose acetate butyrate, polyester and/or an acrylates copolymer, more preferably a nitrocellulose or a polyester or a combination thereof. The concentration of the PFF in the UV gel may range from about 1 wt % to about 20 wt %, preferably from about 2 wt % to about 12 wt %, more preferably about 4 wt % to about 8 wt % relative to the total weight of the UV gel. Inclusion of a PFF typically but not necessarily may also include a solvent to form a homogeneous composition of the PFF in the monomer and multimer components of the UV gel. A plasticizer may also be included with or without the PFF to provide additional flexibility to the cured nail coat.

### Solvent

The UV gel may typically be formed without additional non-reactive solvent. The monomers are liquid under ordinary preparation and use conditions so that they serve a dual function of reactant and solvent. However, at least some additives such as but not limited to the PFF may be appropriately combined with the UV gel monomer and dimer through use of an inert organic solvent including but not limited to one or more C1-C6 monoalcohols, C2-C6 diols, C3-C6 ketones, C1-C3 alkyl acetates, chloroform, ethylene carbonate, propylene carbonate, C5-C10 hydrocarbons and any mixture thereof. Preferably, the organic solvent may be methanol, ethanol, propanol, diacetone alcohol, acetone, methyl ethyl ketone, methyl acetate, ethyl acetate, butyl acetate chloroform, ethylene carbonate, propylene carbonate, hexane, heptane, octane and any combination thereof. The concentration of organic solvent optionally present in the composition may range from about 0.5 wt % to about 15 wt %, preferably from about 0.5 wt % to about 10 wt % relative to the total weight of the UV gel composition.

Pigment and/or Colorant

The particulate-gel composition and the resultant cured nail coat may be colored by incorporation of organic and/or inorganic pigments and/or colorants that are soluble and/or dispersible in inert, volatile organic solvent. The pigment or colorant in solvent may be combined with a portion of the UV gel and/or mixed with the coated glass microparticles and/or combined with the particulate-gel composition. Preferably, the pigment or colorant is combined with the UV gel before its combination with the coated glass microparticles. In any of these combinations, the solvent preferably may be the same non-reactive solvent present with the UV gel and/or coated glass microparticles. After combining, the solvent concentration may be reduced if too high relative to the desired conditions for the UV gel. Preferably, the soluble or dispersible pigment and/or colorant may be combined with the UV gel before adding the coated glass microparticles and the solvent, if any, may be reduced to provide the desired viscosity characteristics for the UV gel. Agitation of the UV gel with pigment and/or colorant such as by stirring, sonic agitation, bladed container rotation or a similar mixing technique will distribute the pigment and/or colorant throughout the UV gel. Combination of the UV gel with the coated glass microparticles before or after addition of the pigment and/or colorant and agitation of the mixture with high speed mixing, or other mixing technique will distribute the pigment and or colorant along with the glass microparticles when the dispersion is formed. The concentration of soluble and/or dispersible pigment and/or colorant may range from 0.1 wt % to 5 wt % relative to the total weight of all components of the particulate-gel composition. The concentration may depend upon the intensity hue and shade of the soluble and/or dispersible pigment and/or colorant to be combined with the other components of the particulate-gel composition.

Organic and inorganic pigments (particulate form) and colorants (soluble form) are well known and available from commercial supply houses. Organic pigments include carbon black, lake types of anionic dye salts, phthalocyanine dyes, quinacridone dyes, dioxazines, isoindolines, perylenes, flavanthrones, anthraquinones, and azo dyes of dinitrogen (—N═N—) cores. Colorants include soluble vegetable dyes such as indigo, sepia, mango, beet, lycopene, red cabbage, turmeric, annatto, beta carotene, paprika, grape and similar vegetable colors. Inorganic pigments include iron oxide, chromium oxide, calcium carbonate, titanium oxide and similar metal compounds. Examples of soluble and/or dispersible pigment include but are not limited to pigment yellow 3, 14, 83, 65; pigment red 53:1, 122, 4; quinacridone, pigment orange 5; pigment blue 29; fast yellow; pigment violet 19 and other similar organic pigments.

### Method for Forming Particulate-Gel Composition

The UV gel may be prepared by combining the monomer and dimer including optional liquid supplemental monomer at appropriate weight percentages and mixed by slow to moderate speed stirring until a clear solution is formed. Additives combined with optional non-reactive organic solvent may then be added and mixing until a uniform gel is prepared. The prepared UV gel may be combined with the coated glass microparticles to form the particulate-gel composition by combining appropriate weight percentages of the UV gel and the coated glass microparticles in a mixing container and high speed mixing at RPM of at least 2000 rotations per minute (RPM) until a dispersion is formed. The high speed mixing may be combined with slight warming and vacuum evaporation to remove the inert organic solvent from the dispersion if needed and/or desired.

Use of larger quantities of coated glass microparticles and UV gel to prepare the particulate-gel composition enable easy apportionment of the particulate-gel composition. Because the resulting dispersion is stable, the larger quantity of particulate-gel composition may be divided into small bottle size portions of so that essentially uniform weight percentages of portions of the coated glass microparticles in each of numerous small units of the particulate-gel composition can be easily produced.

### The Kit

The Kit embodiments according to the invention comprise a single units of the particulate-gel composition. The units may be combined with a cap optionally holding a brush, a sponge, a dispenser and/or an applicator for application of the composition to one or more nail plates.

The Kit may be packaged in a box or other appropriate packaging construct for holding the container unit or units, brushes, sponges, dispensers, applicators and other materials needed for practice of the method according to the invention. Included in the Kit box may also be written instructions for application of the composition to nail plates and UV curing the particulate-gel composition.

The Kit may optionally include a UV lamp designed for curing one or several nails coated with the particulate-gel composition. Typical UV lamps include those configured as handheld units similar to a flashlight or pen light, a lamp with housing designed for irradiation of all fingers by insertion of a hand into housing, a tabletop “C” shape lamp with irradiation fixtures on the top underside and a “C” cavity fitting all fingers of a hand, and a table UV lamp such as a table UV lamp or fixture with a USB attachment for computer control of the irradiation and a flexible neck for adjusting the position of the UV bulb. An exemplary lamp is available through Amazon as a “goose neck” UV lamp.

### The Method of Nail Plate Coating

The nail plate may be stroked, brushed, painted or otherwise contacted by an apparatus holding a portion of the particulate-gel composition to apply this composition to the nail plate and form the composite nail coating. The steps of applying the composition to the nail plate may be repeated to provide additional composite nail coatings on the nail plate.

When one or more applications of composite nail coatings have been applied to the nail plate to provide a desirable completed composite nail coating, the completed composite nail coating may be transformed to cure the UV gel and form the solid coating of film. Curing may be accomplished with a UV lamp for actinic radiation of appropriate wavelength maxima.

### The Cured Nail Coat Or Film

The polymerized solid film on the nail plate comprises the transformed UV gel with dispersed, coated glass microparticles coated on a nail plate, hereinafter the cured nail coat. The cured nail coat of film has high abrasion resistance, high tensile strength and a smooth surface owing at least in part to uniform dispersion of the coated glass microparticles. The resulting cured nail coat may be clear or colored depending on whether pigment and/or colorant is incorporated into the particulate-gel composition.

### Practice of the Method and Experimental Results

In the following experiments, the coated glass microparticles produced by crushing glass sheets are combined with a standard UV Gel, coated on substrate such as a flat, thin polyacrylate sheet, irradiated with UV and the resulting coatings tested for smoothness, tensile strength, abrasion resistance and surface brilliance and shine. Additionally, the experiments can demonstrate the stability of the dispersion of the particulate-gel composition for glass microparticles having a coating and for those not having a coating. Further experiments demonstrate the stability of the particulate-gel composition and resulting cured nail coat properties relative to a range of average longest dimension sizes of the coated glass microparticles.

The experiments include Dispersion Stability Testing of the uncured particulate-gel composition and the Tensile/Yield Strength and Abrasion Resistance of the cured particulate-gel film. These tests may be conducted with calibrated testing devices designed to provide repeatable results. The tests may be conducted according to standard ASTM methods.

### Example UV Gel Composition

The example UV gel was formulated as a mixture of methacrylate dimer and hydroxyalkyl and alkyl methacrylate monomer with preformed film former synthetic polymer such as acrylate copolymer, ethylene-vinyl acetate copolymer and/or nitrocellulose. The monomers act as reactive solvent providing a homogeneous gel having thixotropic properties. The ingredients include di-(methacryloyloxyethylenyl) trimethylhexyl dicarbamate, PEG-9 dimethacrylate, hydroxyethyl methacrylate, hydroxypropyl methacrylate, isobornyl methacrylate, trimethyl benzoyl diphenylphosphine oxide, hydroxycyclohexyl phenyl ketone, BHT, hydroquinole, p-hydoxyanisole, violet 2, silica, mica and optionally at least one of synthetic flurphlogopite, tin oxide, calcium aluminum borosilicate, calcium sodium borosilicate, isophorone diamine/isophthalic acid/tromethamine copolymer, bis(glycidoxyphenyl) propane/bisaminomethylnorbornane copolymer, polybutylene terephthalate, ethylene/vinyl acetate copolymer, calcium titanium bobosilicate, aluminum calcium sodium silicate, alumina, ethanol and/or methyl ethyl ketone solvent; and colorants.

### Coated and Uncoated Glass Microparticles

The coated glass microparticles were purchased from DM Healthcare Products, Inc. The uncoated glass microparticles as spheres were purchased from Cospheric Inc.

### Demonstration of Dispersion Stability

Two experiments were conducted to demonstrate the stability of the coated glass microparticles in standard UV gel relative to the (in) stability of uncoated glass microparticles in the same standard UV gel.

The process for incorporation of the coated glass microparticles and uncoated glass microparticles in standard UV gel was accomplished by combining measured individual aliquots of the microparticles with individual 12 gm portions of the UV gel. Each individual portion of the UV gel was placed in an empty mixing vessel for an ultra high speed mixer, such as a Flacktek SpeedMixer capable of operating at 3500 rpm. Each individual aliquot of uncoated and coated glass microparticles was added to separate UV gel portions in separate individual Flacktek Container Max 20 mixing vessels. Each combined mixture of UV gel and coated or uncoated glass microparticles was stirred at 3000 rpm for 5 minutes to prepare the dispersions of coated and uncoated glass microparticles in the UV gel portions.

The uncoated and coated glass microparticle aliquots for combination with the 50 gm portions of UV gel were measured as indicated on Tables 1 and 2.

TABLE 1
Uncoated Glass Microparticles

| | Glass Particles | | |
|---|---|---|---|
| | Particle Size# | | |
| Loading* | 8-12 μm | 35-45 μm | 65-75 μm |
| 10% | 5 gm | 5 gm | 5 gm |
| 20% | 10 gm | 10 gm | 10 gm |
| 30% | 15 gm | 15 gm | 15 gm |

TABLE 2
Coated Glass Microparticles

| | Glass | | | | | | |
|---|---|---|---|---|---|---|---|
| | | | | | Glass | Glass | |
| | Glass | | | | Micro- | Micro- | |
| | Microparticles | | | | particles | particles | (Cospheric) |
| | H1282 | | | | H1290 | H1344 | P20155L |
| Particle | 1 | 2 | 10 | 65 | 0.7 μm | 2 μm | — |
| Size# | μm | μm | μm | μm | | | |

| | | | | |
|---|---|---|---|---|
| Coating | Trimethoxy- | Trimethoxy- | Trimethoxy- | Uncoated |
| | silylpropyl | silylpropyl | silylpropyl | |
| | Methacrylate | Acrylate | Methacrylate | |

| | |
|---|---|
| Loading | 30% |

*All 30 wt % relative to a 12 gm portion of the UV gel.
#Determined as a statistical average of the largest dimension of a sampling of the glass microparticles

### Results of Dispersion Test

Immediately after high speed stirring for 5 minutes, the Flacktek vessels with the particle-gel compositions (coated and uncoated) were removed from the Flacktek Max 20 mixer and the vessels placed on their sides to determine whether a stable dispersion had been achieved. Overnight, the dispersions with uncoated glass microparticles had separated into extremely thick glass microparticle slurries in residual UV gel in the bottoms of the tipped vessels and almost all of the UV gel in each vessel had flowed out onto the front of the lower side of the tipped vessels. The slurries appeared opaque-white in color while the flowing UV gels were clear and transparent. The tipped vessels containing coated glass microparticles and UV gels showed a viscous opaque dispersion of the coated glass microparticles in the UV gels with no separation of any clear, transparent, very flowable UV gel moving away from the viscous opaque dispersion.

While the uncoated glass microparticle UV gel mixtures separated overnight after stirring, the coated glass microparticle-UV gel mixtures having average longest dimensions from 0.7 microns to 10 microns remained as stable dispersions for up to 4 weeks when contained upright in the stainless steel mixing vessels. Portions of these stable dispersions were poured into polish dispenser bottles and capped to determine whether the size and shape of the vessel would affect the dispersion stability. At the time of the preparation of this application, approximately 11 months, these dispersions have remained stable and homogeneous. The coated glass microparticles of 65 micron size-UV gel mixture were stable for at least a couple of hours but overnight, a substantial amount of the coated glass microparticles separated from the UV gel mixture.

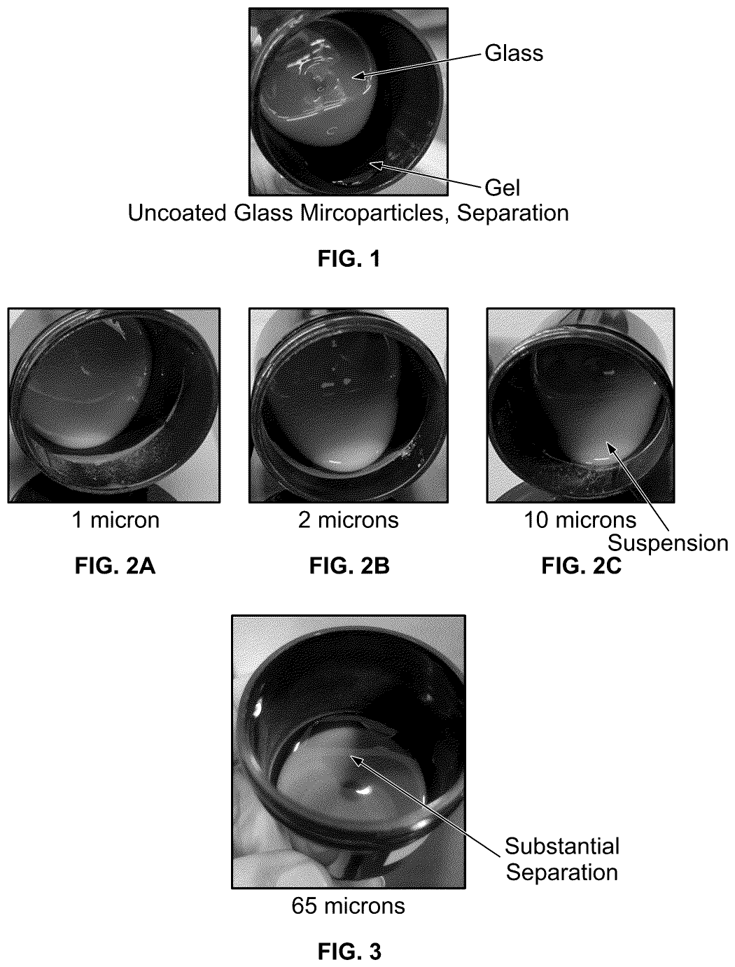

These results are shown in FIGS. 1, 2A, 2B, 2C and 3. FIG. 1 shows the mix cup with uncoated glass microparticles in UV gel in which the microparticles appear as a sludge at the bottom of the cup and the UV gel appears as a clear viscous liquid running to the top of the cup. FIGS. 2A, 2B and 2C show the suspensions of the coated glass microparticles of 1, 2 and 10 microns (mm) in UV gel running to the top of the cups. FIG. 3 shows the substantially separated condition of coated glass microparticles of 65 microns in UV gel. The separated 65 micron coated glass microparticles appear as sludge overcoated by clear UV gel at the upper bottom end of the cup. At least some of the 65 micron coated microparticles are still suspended as the appearance at the middle portion of the cup. Together these experiments demonstrate that alkyl silicone coated glass microparticles of about a 45 micron size will remain suspended in UV gel.

### Viscosity Determinations

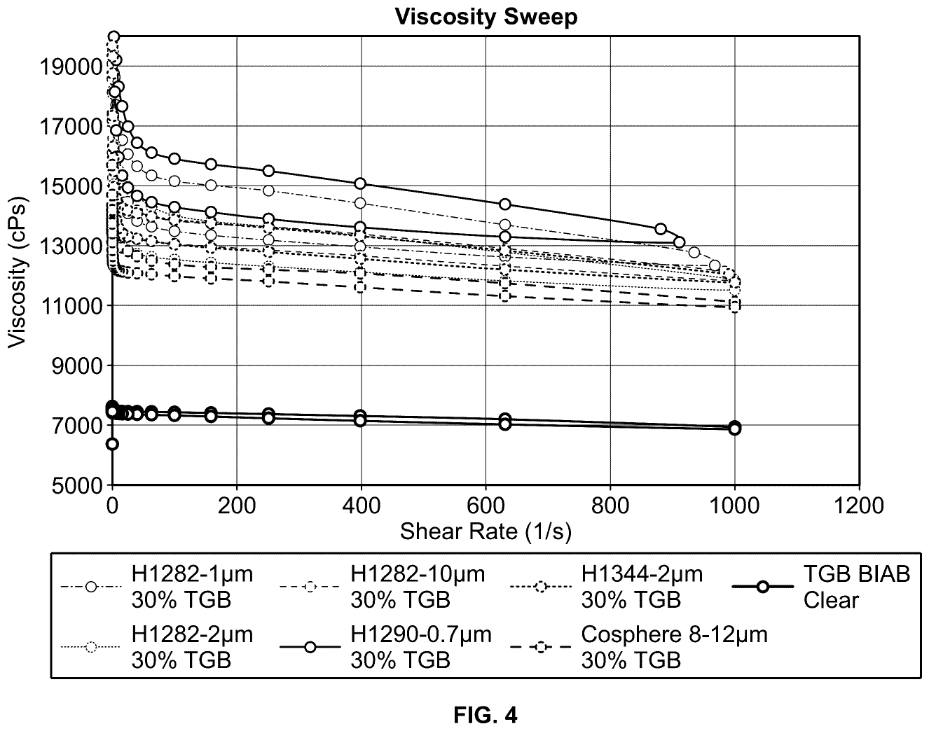

Viscosity determinations of the stable dispersions described above on Table 2 were conducted using a rheometer. Relative to the viscosity of the UV gel at rest, the compositions with coated glass microparticles (particulate-gels) showed significantly increased viscosity at rest. Under shear conditions, the particulate-gels showed thixotropic properties while the UV gel alone did not. FIG. 4 presents a graph of these results and demonstrates this difference. TGB is an acronym for the “the gel bottle” in which the BIAB or UV gel builder in a bottle as described above for a UV gel with a viscosity of the UV gel alone of about 7200-7600 cP viscosity as indicated by the solid circle/bold graph line of FIG. 4.

Abrasion Test of Cured UV Gel with Coated Glass Microparticles

The “Gardner Wet Abrasion method” was used to test the abrasion resistance of cured UV gel with a) coated glass microparticles (sample a) and b) UV gel alone (sample b) see www.byk-instruments.com; Article No. 5060, Gardner-scrub, base. The glass microparticle size for a) was 2 microns. Sample a) were prepared by high speed shear mixing as described above.

BYK Leneta Cards of long length were used as the substrate on which sample (a) and sample (b) were coated. The Leneta Card used was a 14 mil thick paperboard with a sealed black coating on its top side for composition application. A 3 mil drawdown of each sample was made side-by-side on the Cards. Each Card was marked to identify the individual sample applied. The coatings were exposed to UV radiation having wavelength maxima at about 320-420 with lamp maxima at 365 and 405 nm according to the absorption maxima of the photoinitiators of the UV gel to activate the photoinitiators and obtain surface and depth polymerization at the same time. Each sample was exposed for 30 seconds to assure full polymerization and produce a cured film on the Leneta Cards. Each Card was examined for imperfections, confirmation of lack of tackiness, smoothness and brilliance of appearance. The Cards were scanned on a Xerox Altalink CX055 to determine their mean gray values. The gray value is a measure of the black shade transmitted through the coating on the black Leneta Card. The Standard Leneta Card black background has an average mean gray value of 31. The lower the average gray value, the blacker is the detected surface. For example, an average gray value of 40 for coating on this kind of Leneta Card means that less of the black color of the card is reflected through the coating.

Following preparation and examination of the samples, each cured Card was placed on the BYK Gardner-scrub machine base model 5060. The machine was set up with BYK scrubbing media and brush to perform the scrub test. The brush scrub was conducted 2000 cycles for each Card. The scrubbing brushes were positioned at an end section of each the coated, cured Card and occupied approximately ⅓ to ½ of the distance between the lateral edges of each card. The scrubbing brushes were then conveyed from one end to the other of each card so as to produce a scratch strip from one to the other of each card. The resulting brush pattern on each card was a longitudinal strip from one end to the other of each card. The scrubbed Cards were removed from the machine washed and wiped clean. The coated Cards were scanned again with the Xerox Altalink CX055 machine to determine their grayness scores. The Altalink CX055 scanning process provides an indication of the grey shade transmitted by the black Leneta Card. The grey scale runs from no light reflected as black to all light reflected as white. The darker or blacker the shade, the less light is reflected to the scanning detector.

Before conducting the brush scrub, Sample A with coated glass microparticles in UV gel had high mean gray value of 42.1. Before conducting the brush scrub, Sample B with UV gel alone had lower mean gray value 34.8. A gray value number of these “before scrubbing” results that is higher than the Leneta Card standard value of 31 means that an increased degree (lumens) of light is reflected by the cured coating rather than being transmitted through the cured coating and absorbed by the black surface of the Leneta Card.

After the 2000 cycle scrub test, sample A had average mean gray value of 42 while sample B had average mean gray value of 39.5. This shows that there was no change in sample A which indicates that sample A was not scratched. Sample B was abraded or scratched so that more scanning light was reflected by sample B after scrubbing than before scrubbing.

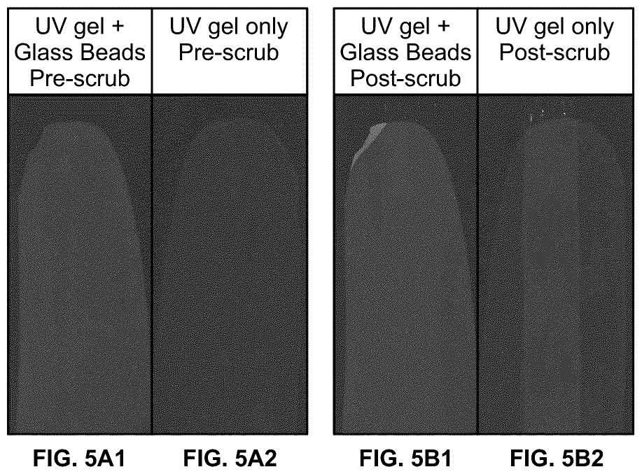

The before and after scanning values for these test samples are charted on the following Table 3. The before and after scrubbing Leneta Cards are shown in FIGS. 5A1, 5A2, 5B1 and 5B2. FIGS. 5A1 and 5B1 show the before and after Leneta Cards for the cured UV gel plus coated glass microparticles. The greyness of 5A1 and 5B1 are the same indicating no scratching by the 2000 cycles of scrubbing occurred for this embodiment of the invention. FIGS. 5A2 and 5B2 show the before and after Leneta Cards for the cured UV gel alone. The lighter strip along the left side of FIG. 5B2 shows that the UV gel alone was significantly scratched by the 2000 cycles of scrubbing.

TABLE 3

| | Mean Average |
|---|---|
| Sample | Gray Value |
| UV gel + Glass Beads | 42.1 |
| Pre-Scrub | |
| UV gel + Glass Beads | 42.0 |
| Post-Scrub | |
| UV gel Pre-scrub | 34.8 |
| UV gel Post-scrub | 39.5 |
| Black Lineta Card | 31.0 |
| Blank Reference | |

### The Statements of Embodiments of the Invention

The Statements of the Invention set forth further Embodiments of the Invention and provide details described above as well as additional details according to the Invention. The Statements supplement and expand the description of the plastic extension embodiments set forth in the Detailed Description. Discrepancies between the Statements and Detailed Description are to be considered to be additive rather than subtractive.

### Statement 1A

1A. A particulate-gel composition comprising a stable dispersion of coated glass microparticles in a UV gel wherein:
the coated glass microparticles comprise glass microparticles having a longest average dimension of from about 0.01 microns to about 45 microns which are coated with residues of an alkoxysilyloxy alkyl (meth)acrylate mono-, di- or tri-(C1-C3 alkoxy) silyloxy C1-C3 alkyl (meth)acrylate;
the UV gel comprises at least one photoinitiator and at least one or more unsaturated carboxylate compounds wherein the one or more carboxylate compounds comprise (meth)acrylate and/or crotonate monomers alone, or one or more (meth)acrylate and/or crotonate multimers alone, or a combination of the one or more (meth)acrylate and/or crotonate monomers and one or more (meth)acrylate and/or crotonate multimers.

### Statement 1B

1B 1A. A particulate-gel composition comprising a stable dispersion of coated glass microparticles in a UV gel wherein:
the coated glass microparticles comprise glass microparticles of have a longest average dimension of from about 0.01 microns to about 45 microns which are coated with residues of a mono-, di- or tri-(C1-C3 alkoxy) silyloxy C1-C3 alkyl (meth)acrylate;
the UV gel comprises
   a) one or more (meth)acrylate and/or crotonate ester monomers alone wherein the esterifying alcohol is one or more residues of a C1-C20 alkanol, preferably a C1-C15 alkanol, more preferably a C1-C10 alkanol, especially more preferably an C1-C6 alkanol, an α,ω-dihydroxy C2-C20 alkane, preferably α,ω-dihydroxy C2-C15 alkane, more preferably α,ω-dihydroxy C2-C10 alkane, especially more preferably α,ω-dihydroxy C2-C6 alkane, or a C5-C16 cycloalkanol, preferably a C5-C14 cycloalkanol, more preferably a C5-C12 cycloalkanol, especially more preferably a C5-C10 cycloalkanol, isobornyl alcohol, hydroxycyclohexane or any combination thereof; or
   b) one or more (meth)acrylate and/or crotonate multimers alone wherein the one or more multimers comprises an ester oligomer or urethane oligomer or amide oligomer or polyol oligomer or dimethyl siloxane oligomer having two, three or four terminal branches wherein each branch is terminated with (meth)acrylate and/or crotonate; or
   c) a combination of the one or more (meth)acrylate and/or crotonate monomers of a) and one or more (meth)acrylate and/or crotonate multimers of b), and,
   d) one or more photoinitiators.

1c. The particulate-gel composition according to statement 1a or 1b wherein the UV gel comprises a viscosity of from about 2K cP to about 500K cP, preferably from about cP to about 450K cP, more preferably from about 2K cP up to about 350K to about 400K cP.

2. The particulate-gel composition according to statement 1a, 1b or 1c wherein:
a) the one or more (meth)acrylate and/or crotonate ester monomers comprise one or more of methyl (meth)acrylate, ethyl (meth)acrylate, isobutyl (meth)acrylate, hydroxyethyl (meth)acrylate, hydroxypropyl (meth)acrylate, isorbornyl (meth)acrylate, cyclohexyl (meth)acrylate or any combination thereof; and,
b) the one or more (meth)acrylate and/or crotonate multimers comprise one or more of an α,ω,ω-trihydroxy or α,ω dihydroxy-2 to 20 unit C3-C9 alkyl urethane oligomer triesterified or diesterified respectively by (meth)acrylic acid, or an α,ω,ω-triamino or α,ω-diamino-2 to 20 unit C3-C6 alkyl amide oligomer triamidated or diamidated respectively by (meth)acrylic acid, or a 2 to 20 unit trihydroxy or dihydroxy poly C2-C6 alkylene oxide triesterified or diesterified respectively by (meth)acrylate, or any combination thereof.

3. The particulate-gel composition according to statement 1a, 1b, 1c or 2 wherein the one or more (meth)acrylate multimers are 1,6-dihydroxyethyl-2,2,4-trimethyl hexyl dicarbamate diesterified by (meth)acrylate, or di or tri-ethylene or propylene oxide diesterified by (meth)acrylate or trimethylol propane triesterified by (meth)acrylate or a combination thereof.

4. The particulate-gel composition according to statement 1a, 1b, 1c, 2 or 3 wherein the one or more photoinitiators are benzophenone, phosphine oxide, TPO, TPO-L or 1-hydroxycyclhexylphen ketone or any combination thereof.

5. The particulate-gel composition according to any of the preceding claims wherein the coating of the glass microparticles is a residue of the mono-, di- or tri-(C1-C3 alkoxy) silyloxy C1-C3 alkylenyl (meth)acrylate of the Formula II

(R¹O)ₙ(R²)₃₋ₙSiO—R³—O₂CC(R⁴)═CH₂ Formula II
wherein R¹ is methyl or ethyl, R² is methyl, R³ is methylenyl, ethylenyl or propylenyl, R⁴ is H or methyl and designator n is an integer of 1, 2 or 3.

6. The particulate-gel composition according to statement 5 wherein n is 3, R¹ is methyl, R³ is ethyl and R⁴ is H.

7. The particulate-gel composition according to any of the preceding statements wherein the UV gel comprises a mixture of hydroxyethyl acrylate, methyl methacrylate, isobornyl acrylate, a (meth)acrylate multimer comprising one or more of an α,ω-dihydroxy-2-20 unit C3-C9 alkyl urethane oligomer di or tri esterified with (meth)acrylate or an α,ω dihydroxy 2-20 unit ethylene or propylene oxide oligomer di or tri esterified with (meth)acrylate.

8. The particulate-gel composition according to any of the preceding statements wherein the (meth)acrylate multimer comprises a mixture of a low unit number fast curing oligomeric multimer and a high unit number slow curing oligomeric multimer.

9. The particulate-gel composition according to statement 8 wherein the fast curing oligomer is a 1 to 4 unit oligomer and the slow curing oligomer is a 5 to 20 unit oligomer.

10. The particulate-gel composition according to statement 9 wherein the mol ratio of the fast curing multimer to slow curing multimer is 1:2 to 1:5.

11. The particulate-gel composition according any of the preceding statements wherein the coated glass microparticles have an average dimension range of from about 0.1 micron to about 45 microns, preferably from about 0.1 microns to about 30 microns, more preferably from about 0.1 microns to about 20 microns, especially more preferably from about 0.1 microns to about 10 microns.

12. The particulate-gel composition according to any of the preceding statements wherein the weight percent of coated glass microparticles in the particulate-gel composition is from about 1 wt % to about 60 wt %, preferably from about 2 wt % to about 50 wt %, more preferably from about 5 wt % to about 40 wt %, especially more preferably from about 10 wt % to about 35 wt % relative to the total weight of the particulate-gel composition.

13. The particulate-gel composition according to any of the preceding statements wherein the UV gel further comprises an ancillary agent comprising one or more of a rheology controller, a free radical scavenger, an oxygen scavenger, a thixotrope, a plasticizer, a gel spreader, a color, a pigment, an anionic, cationic and/or nonionic surfactant, a cationic polymer, a supplemental particulate clay or silica, a hydroquinone, a preformed film former, an organic solvent or any combination thereof.

14. The particulate-gel composition according to statement 13 wherein the ancillary agent comprises a soluble and/or particulate pigment and/or colorant, a gum selected from locust gum, guar gum, acacia gum, xanthan gum and a natural vegetable thickening agent; a cationic polymer selected from trimethylammonium alkyl cellulose, poly(trimethyl ammonium propyl (meth)acrylate); a supplemental particulate selected from silica, mordenite and/or bentonite and/or hectorite clay; one or more anionic and/or cationic and/or nonionic surfactants selected from lauryl sulfonate, laureth sulfonate, lauryl carboxylate, a trimethylammonium halide of a fatty C10-C26 alkyl/alkenyl group selected from behenyl, cetyl, stearyl, oleyl, myristyl or palmitoleyl; one or more plasticizers selected from a phthalate ester, a trimellitate ester, an adipate ester or a dialkyl oligoglycol, sucrose benzoate; a polysiloxane selected from a dimethicone or a polysilicone or a silicone copolymer with ethylene oxide and/or propylene oxide oligomer or polymer block units; hydroquinone, 4-methoxylphenol, an oxygen scavenger, an adhesion promoting primer selected from methacrylic acid, ethyl acetate and/or an organic solvent.

15. The particulate-gel composition according to statement 13 or 14 wherein the ancillary agent further comprises a preformed film former (PFF) comprising nitrocellulose, ethyl cellulose, cellulose acetate, cellulose acetate butyrate, tosylamide epoxy resin, acrylates copolymer, a polyester, a stryrene/acrylates copolymer, an adipic acid/neopentylglycol/trimellitic anhydride copolymer and/or any mixture thereof.

16. The particulate-gel composition according to any of statements 13-15 wherein the UV gel further comprises a non-reactive organic solvent wherein the solvent is a C2-C4 alkanol, acetone, methyl ethyl ketone, a C6-C8 hydrocarbon or any combination thereof.

17. The particulate-gel composition according to statement 15 wherein the PFF enables the formation of a two phase solid with the cured UV gel wherein the cured UV gel is a continuous phase and the PFF is a discontinuous phase and the PFF discontinuous phase enables easy, mild removal of the cured nail coat by an organic solvent selected from acetone, methyl ethyl ketone or a mixture thereof.

18. The particulate-gel composition according to any of statements 15-17 wherein the concentration of the PFF in the UV gel may range from about 1 wt % to about 20 wt %, preferably from about 2 wt % to about 15 wt % relative to the total weight of the UV gel.

19. The particulate-gel composition according to any of statements 1a, 1b or 1c-18 wherein the coated glass microparticles have a regular or irregular shape or a combination thereof.

20. A method for preparing the particulate-gel composition of any of the preceding statements comprising combining the coated glass microparticles and the UV gel with a high speed blending.

21. The method according to statement 20 wherein the high speed blending is high shear mixing.

22. A method for applying the particulate-gel composition of any of statements 1a, 12b, 1c-19 to a substrate comprising contacting the substrate with one or more portions of the particulate-gel composition to produce a composite coat.

23. The method according to statement 22 wherein the substrate is a nail plate of a fingernail and the composite coat is a composite nail coat.

24. The method according to statement 22 or 23 wherein the contacting is accomplished by spraying, dripping, brushing, layer flow stream, bead or absorbent pad delivery.

25. The method according to any of statements 22-24 wherein the contact is accomplished by one or more strokes of an applicator, spray, brush, tube or dispenser.

26. A method for preparing a cured nail coat comprising UV irradiating the composite nail coat of statement 23.

27. A cured nail coat comprising a particulate-gel composition according to any of the preceding statements 1a, 1b, 1c-19 on a nail plate of a fingernail irradiated by UV irradiation.

28. A cured nail coat on a substrate prepared according to the method of statement 26.

29. The cured nail coat according to any of statements 27-28 displaying a smooth surface and surface abrasion resistance.

30. Use of a particulate-gel composition according to any of statements 1a, 1b, 1c-19 to produce a cured nail coat on a substrate.

31. Use according to statement 30 wherein the substrate is a nail plate.

32. A kit comprising the particulate-gel composition according to any of statements 1a, 1b, 1c-19 in a unit container.

33. The kit according to statement 32 wherein the unit container is a bottle with a cap holding a brush or applicator.

34. The kit according to statement 32 or 33 wherein the unit container with brush, applicator, dispenser and/or tubular orifice and particulate-gel composition is adapted to provide a fingernail covering.

35. The kit according to any of statements 32-34 further comprising a UV lamp.

36. The kit according to statement 36 wherein the UV lamp is a table UV lamp for irradiating at least one finger.

### SUMMARY STATEMENTS

The inventions, examples and results described and claimed herein may have attributes and embodiments include, but not limited to, those set forth or described or referenced in this application.

All patents, publications, scientific articles, web sites and other documents and ministerial references or mentioned herein are indicative of the levels of skill of those skilled in the art to which the invention pertains, and each such referenced document and material is hereby incorporated by reference to the same extent as if it had been incorporated verbatim and set forth in its entirety herein. The right is reserved to physically incorporate into this specification any and all materials and information from any such patent, publication, scientific article, web site, electronically available information, textbook or other referenced material or document.

The written description of this patent application includes all claims. All claims including all original claims are hereby incorporated by reference in their entirety into the written description portion of the specification and the right is reserved to physically incorporated into the written description or any other portion of the application any and all such claims. Thus, for example, under no circumstances may the patent be interpreted as allegedly not providing a written description for a claim on the assertion that the precise wording of the claim is not set forth in haec verba in written description portion of the patent.

While the invention has been described in conjunction with the detailed description thereof, the foregoing description is intended to illustrate and not limit the scope of the invention, which is defined by the scope of the appended claims. Thus, from the foregoing, it will be appreciated that, although specific nonlimiting embodiments of the invention have been described herein for the purpose of illustration, various modifications may be made without deviating from the scope of the invention. Other aspects, advantages, and modifications are within the scope of the following claims and the present invention is not limited except as by the appended claims.

1. A particulate-gel composition comprising a stable dispersion of coated glass microparticles in a UV gel wherein:
the coated glass microparticles comprise glass microparticles having a longest average dimension of from about 0.01 microns to about 45 microns which are coated with residues of an alkoxysilyloxy alkyl (meth)acrylate mono-, di- or tri-(C1-C3 alkoxy) silyloxy C1-C3 alkyl (meth)acrylate;
the UV gel comprises at least one photoinitiator and one or more unsaturated carboxylate compounds comprising one or more (meth)acrylate and/or crotonate monomers alone, or one or more (meth)acrylate and/or crotonate multimers alone or a combination of one or more (meth)acrylate and/or crotonate monomers and one or more (meth)acrylate and/or crotonate multimers.

2. The particulate-gel composition according to claim 1 wherein
a) the one or more (meth)acrylate and/or crotonate monomers alone comprises one or more (meth)acrylate ester monomers wherein the esterifying alcohol is one or more residues of a C1-C20 alkanol, preferably a C1-C15 alkanol, more preferably a C1-C10 alkanol, especially more preferably an C1-C6 alkanol, an α,ω-dihydroxy C2-C20 alkane, preferably α,ω-dihydroxy C2-C15 alkane, more preferably α,ω-dihydroxy C2-C10 alkane, especially more preferably α,ω-dihydroxy C2-C6 alkane, or a C5-C16 cycloalkanol, preferably a C5-C14 cycloalkanol, more preferably a C5-C12 cycloalkanol, especially more preferably a C5-C10 cycloalkanol, isobornyl alcohol, hydroxycyclohexane or any combination thereof;
b) the one or more (meth)acrylate and/or crotonate multimers alone comprise one or more of a (meth)acrylate multimer comprising an ester oligomer or urethane oligomer or amide oligomer or polyol oligomer or dimethyl siloxane oligomer having two, three or four terminal branches wherein each branch is terminated with (meth)acrylate; and,
c) the combination of one or more (meth)acrylate and/or crotonate monomers and one or more (meth)acrylate and/or crotonate comprise a combination of a) and b).

3. The particulate-gel composition according to claim 1 wherein the coating of the glass microparticles is a residue of the mono-, di- or tri-(C1-C3 alkoxy) silyloxy C1-C3 alkylenyl (meth)acrylate of the Formula II
(R¹O)ₙ(R²)₃₋ₙSiO—R³—O₂CC(R⁴)═CH₂ Formula II
wherein R¹ is methyl or ethyl, R² is methyl, R³ is methylenyl, ethylenyl or propylenyl, R⁴ is H or methyl and designator n is an integer of 1, 2 or 3.

4. The particulate-gel composition according to claim 1 wherein the coated glass microparticles have a regular or irregular shape or a combination thereof.

5. The particulate-gel composition according to claim 1 wherein the unsaturated carboxylate monomer comprises hydroxyethyl acrylate, methyl methacrylate, isobornyl acrylate, the unsaturated carboxylate multimer comprises a (meth)acrylate multimer of one or more of an α,ω-dihydroxy-1-20 unit C3-C9 alkyl urethane oligomer diesterified with (meth)acrylate or an α,ω dihydroxy 1-20 unit ethylene or propylene oxide oligomer diesterified with (meth)acrylate.

6. The particulate-gel composition according to claim 1 wherein the weight percent of coated glass microparticles in the particulate-gel composition is from about 1 wt % to about 60 wt %, preferably from about 2 wt % to about 50 wt %, more preferably from about 5 wt % to about 40 wt %, especially more preferably from about 10 wt % to about 35 wt % relative to the total weight of the particulate-gel composition.

7. The particulate-gel composition according to claim 1 wherein the UV gel further comprises an ancillary agent comprising one or more of a rheology controller, a free radical scavenger, an oxygen scavenger, a thixotrope, a plasticizer, a gel spreader, a color, a pigment, an anionic, cationic and/or nonionic surfactant, a cationic polymer, a supplemental particulate clay or silica, a hydroquinone, a preformed film former, an organic solvent or any combination thereof.

8. The particulate-gel composition according to claim 1 wherein the UV gel further comprises a preformed film former (PFF) comprising nitrocellulose, ethyl cellulose, cellulose acetate, cellulose acetate butyrate, tosylamide epoxy resin, acrylates copolymer, a poly methyl (meth)acrylate, a poly (meth)acrylate, a polyester, a stryrene/acrylates copolymer, an adipic acid/neopentylglycol/trimellitic anhydride copolymer and/or any mixture thereof.

9. The particulate-gel composition according to claim 1 wherein the UV gel has a viscosity of from about 2K cP to about 500K cP, preferably from about 2K cP to about 450K cP.

10. A method for preparing the particulate-gel composition comprising combining the coated glass microparticles and the UV gel of claim 1.

11. A kit comprising the particulate-gel composition according to claim 1 in a unit container and a brush or applicator.

12. The kit according to claim 11 further comprising a UV lamp.

13. A unit container with a particulate-gel composition of claim 1 wherein the particulate-gel composition is a stable suspension for at least 4 months, preferably for at least 12 months.

14. A method for preparing a cured nail coat on a fingernail comprising applying the particulate-gel composition of claim 1 to the nail plate of the fingernail to form a composite nail coat and irradiating the composite nail coat with actinic radiation to produce the cured nail coat.

15. A cured nail coat on a nail plate prepared according to the method of claim 14.

16. A coating a nail plate of a fingernail wherein the coating comprises a cured particulate-gel composition of claim 1.
